(19) United States
(12) Patent Application Publication
(10) Pub. No.: US 2022/0061815 A1
(43) Pub. Date: Mar. 3, 2022

(54) MEASURING PHYSIOLOGICAL MOTION USING FMCW RADAR

(71) Applicant: Zachary Flaherty, Rancho Mirage, CA (US)

(72) Inventor: Zachary Flaherty, Rancho Mirage, CA (US)

(21) Appl. No.: 17/010,116

(22) Filed: Sep. 2, 2020

(51) Int. Cl. A61B 8/08; G01S 7/40

(52) U.S. Cl. CPC A61B 8/488; G01S 13/505; G01S 7/4056

(57) ABSTRACT

Systems and methods for monitoring vital signs (e.g. heartbeat, respiration) using FMCW millimeter wave radar are disclosed herein. A transceiver is used to transmit a first signal (FMCW) and receive a second signal (reflected). The transceiver transmits the second signal data to a computing device. A first set of radar data is generated by software on the computing device, based on the received second signal. A first set of Doppler interval measurements is obtained from the first set of radar data. A high Doppler response is obtained from the first set of Doppler interval measurements and vital sign data is extracted from the high Doppler response. Advantages include the use of Doppler frequencies which are free to use according to FAA specifications; living organisms (subjects) are not affected by the radiation or the transmission path; and a subject may be remotely monitored without requiring physical access.

### CROSS-REFERENCE TO RELATED APPLICATIONS

This is a Nonprovisional Utility U.S. patent application under 37 CFR 1.53(b).

### BACKGROUND OF THE INVENTION

### Field of the Invention

The present invention relates generally to the monitoring of the physiological movement of subjects. More particularly, the present invention relates to devices and methods using FMCW radar and software algorithms to monitor vital signs of subjects.

### Description of Related Art

“RADAR” stands for Radio Detection and Ranging, meaning that it is capable of detecting objects and also evaluating object parameters. Radar systems have long been employed for determining the distance between a target and the radar system. The distance is directly related to a transmission of a frequency modulated signal and a received reflected frequency modulated signal. The distance is calculated by taking the time delay or frequency difference between the transmitted and received signals. Radar systems use transceivers, which can include a radio frequency antenna for transmitting and a separate receiver antenna for receiving the reflected signal.

Traditionally, the presence and motion of objects were detected using continuous wave (CW) Doppler radar. The CW Doppler radar is a simple and efficient solution only when detection of a moving object is the only outstanding task to be completed. CW Doppler radar uses the Doppler effect, which concerns all sorts of wave generators and states the following:

Wave fronts, transmitted by a wave generator (e.g. sound, microwaves, light, etc.) hit a moving target. Depending on the direction of the motion of the object, the wave fronts are either “compressed” or “diluted”, which means a shift in frequency. The signal, shifted in frequency and reflected, is subtracted from the unchanged transmit signal in a relatively simple mixer (called “homodyne” mixing) and results in a sinusoidal intermediate frequency (IF). It is irrelevant whether the sensor moves relatively to the object or the object moves relatively to the sensor. Only the component of the velocity vector pointing parallel to the direct connection sensor-object can be calculated. The speed of an object can be evaluated by measuring the Doppler frequency, while considering the angle of the motion vector. Measuring the Doppler frequency is done by counting the zero crossings in an analog system or using fast Fourier Transforms (FFT) in a digital system.

A traditional method to measure position and distance (range) of an object involves using frequency modulated continuous wave (FMCW) “pulse” radar. A time delay is generated and measured by transmitting a short pulse and clocking the reception of the reflected pulse. Since the transmitted pulse and reflected pulse travel at the speed of light, a pulse would be delayed by six nanoseconds for an example object distance of one meter. There are significant issues with using pulse radar. For example, in order to obtain good resolution for an object at a close distance away, the pulses must be very short. Short pulses require enormous bandwidth, which may not be allowed by certification and regulation authorities such as the Federal Aviation Administration (FAA). In general, pulse radar is primarily used to evaluate the distance and range of objects. Velocity information can only be obtained by a timely derivation (ds/dt) of distance over time taken from a plurality of measured distance values.

When a bacterial or viral infection is present, human and non-human animal (e.g. cattle) subjects have symptoms such as elevated heart rate and elevated respiration. Medical professionals are often notified of an infection only after a fever or other discomfort has developed at an aggravated state of the infection. There is a need in the field for a system and method to continuously monitor and store a history of vital signs of a subject prior to an infection reaching an aggravated state, thereby enabling a determination of how the infection initially occurred.

### SUMMARY OF THE INVENTION

Systems and methods for monitoring vital signs (e.g. heartbeat, respiration) using FMCW millimeter wave radar are disclosed herein. A transceiver is used to transmit a first signal (FMCW) and receive a second signal (reflected). The transceiver transmits the second signal data to a computing device. A first set of radar data is generated by software on the computing device, based on the received second signal. A first set of Doppler interval measurements is obtained from the first set of radar data. A high Doppler response is obtained from the first set of Doppler interval measurements and vital sign data is extracted from the high Doppler response.

There are several advantages of the present invention, including the use of Doppler frequencies which are free to use according to FAA specifications. The operating frequencies are very high and living organisms (subjects) are not affected by the radiation or the transmission path. The FMCW radar system and method enables a subject to be remotely monitored without requiring physical access to the subject. Wireless transmission is utilized to transmit signal data from a transceiver to a computing device with software installed.

These and other features and advantages will be apparent from reading of the following detailed description and review of the associated drawings. It is to be understood that both the forgoing general description and the following detailed description are explanatory and do not restrict aspects as claimed.

### BRIEF DESCRIPTION OF THE DRAWINGS

FIG. 1 illustrates a flow diagram of a system and method for measuring physiological motion of a subject using FMCW radar.

FIG. 2 illustrates a flow diagram of a system and method for measuring physiological motion of a subject using FMCW radar and complex software algorithms.

FIG. 3 illustrates a graph depicting changes in frequency over time for a FMCW radar system.

FIG. 4 illustrates a diagram of a system and method for measuring physiological motion of a human subject.

FIG. 5 illustrates a diagram of a system and method for measuring physiological motion of an animal, namely cattle.

### DETAILED DESCRIPTION OF EMBODIMENTS

The following descriptions relate principally to preferred embodiments while a few alternative embodiments may also be referenced on occasion, although it should be understood that many other alternative embodiments would also fall within the scope of the invention. The embodiments disclosed are not to be construed as describing limits to the invention, whereas the broader scope of the invention should instead be considered with reference to the claims, which may be now appended or may later be added or amended in this or related applications. Unless indicated otherwise, it is to be understood that terms used in these descriptions generally have the same meanings as those that would be understood by persons of ordinary skill in the art. It should also be understood that terms used are generally intended to have the ordinary meanings that would be understood within the context of the related art, and they generally should not be restricted to formal or ideal definitions, conceptually encompassing equivalents, unless and only to the extent that a particular context clearly requires otherwise.

For purposes of these descriptions, a few wording simplifications should also be understood as universal, except to the extent otherwise clarified in a particular context either in the specification or in particular claims. The use of the term “or” should be understood as referring to alternatives, although it is generally used to mean “and/or” unless explicitly indicated to refer to alternatives only, or unless the alternatives are inherently mutually exclusive. Furthermore, unless explicitly dictated by the language, the term “and” may be interpreted as “or” in some instances. When referencing values, the term “about” may be used to indicate an approximate value, generally one that could be read as being that value plus or minus half of the value. “A” or “an” and the like may mean one or more, unless clearly indicated otherwise. Such “one or more” meanings are most especially intended when references are made in conjunction with open-ended words such as “having,” “comprising” or “including.” Likewise, “another” object may mean at least a second object or more. Thus, in the context of this specification, the term “comprising” is used in an inclusive sense and thus should be understood as meaning “including, but not limited to.” As used herein, the use of “may” or “may be” indicates that a modified term is appropriate, capable, or suitable for an indicated capacity, function, or usage, while considering that in some circumstances the modified term may sometimes not be appropriate, capable, or suitable. A “computing device” can be a desktop, laptop, tablet, phone, and the like.

The disclosed FMCW radar systems and methods can be used for detecting and evaluating motion of objects (e.g. human or animal subjects, vehicles, machines). In a case of a stationary object, FMCW radar can define the object's instantaneous position (range, angle). In a case of a moving object, FMCW radar can measure the object's movement (velocity, direction) and clock and track the continuously changing position. FMCW radar uses high frequency electromagnetic (EM) waves to provide sufficient resolution and to accurately evaluate objects.

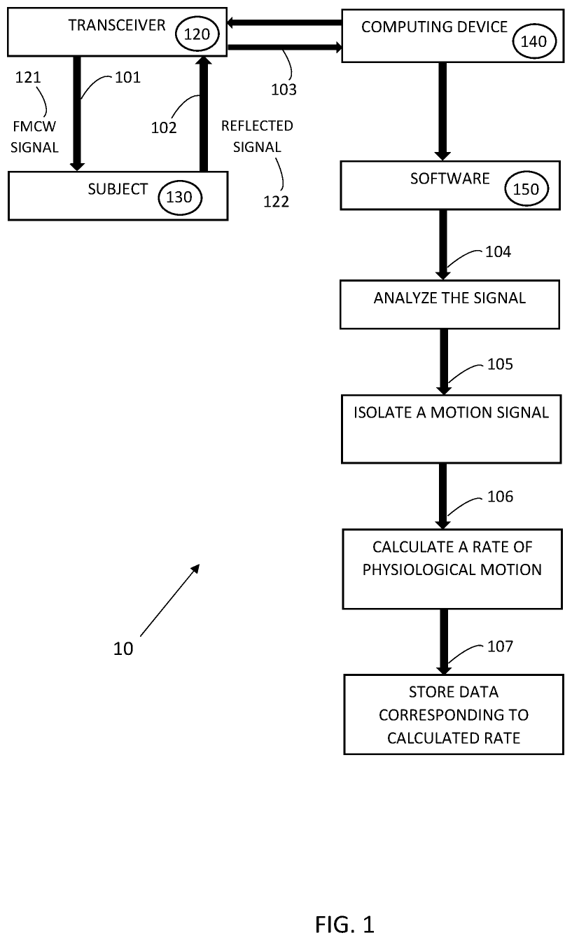

FIG. 1 illustrates a flow diagram 10 of a system and method for measuring physiological motion of a subject using FMCW radar. In step 101, a transceiver 120 transmits a first signal 121 (FMCW signal) toward a subject 130, which can be a human or other animal. The transceiver 120 can include a radio frequency (RF) transmitting antenna and a receiver antenna. A computing device 140 in communication with the transceiver 120 can be used to initiate the transmission 101 of the FMCW signal. In step 102, a second signal 122 (reflected signal) from the subject 130 is received by the transceiver 120. In step 103, the transceiver transmits the second signal 122 data to the computing device 140. The transmission 103 can occur via a hard-wiring system or a wireless system such as WiFi, Bluetooth, and the like. In step 104, software 150 installed on the computing device analyzes the second signal 122 data to determine types of physiological motion measured of the subject 130. In step 105, the software 150 isolates a motion signal corresponding to physiological motion of the subject 130. The physiological motion detected can be a heartbeat, respiration, or another motion of a subject 130. For example, during respiration there is displacement of the chest and thorax of a subject 130. Similarly, there is motion associated with a heartbeat of a subject 130. In step 106, the software 150 calculates a rate of physiological motion of the subject 130 based on the motion signal. For example, a rate of physiological motion for a heartbeat can be calculated in beats per minute. In step 107, the software 150 stores data corresponding to the calculated rate 106 of the physiological motion of the subject 130.

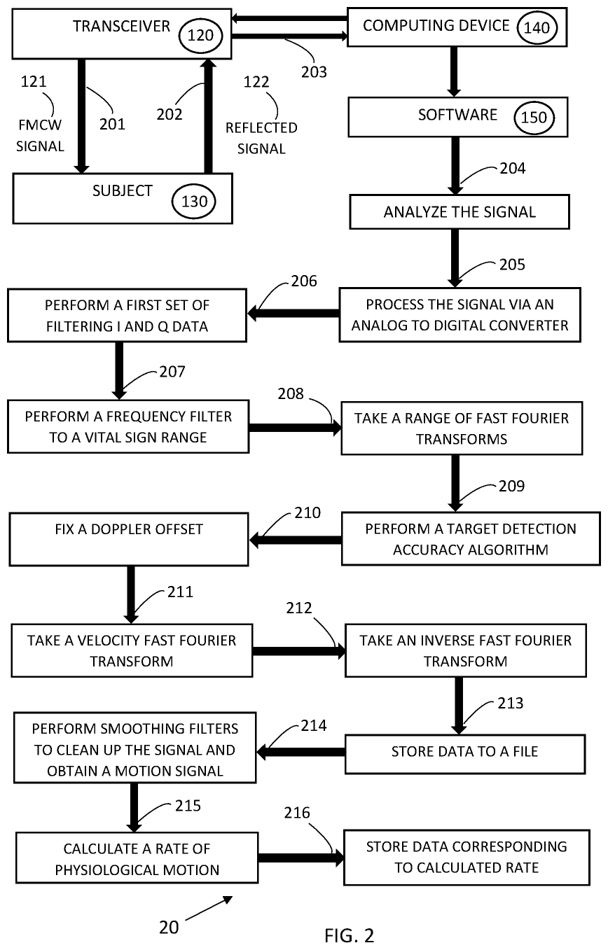

FIG. 2 illustrates a flow diagram 20 of a system and method for measuring physiological motion of a subject using FMCW radar and complex software algorithms. In step 201, a transceiver 120 transmits a first signal 121 (FMCW signal) toward a subject 130, which can be a human or other animal. The transceiver 120 can include a RF transmitting antenna and a receiver antenna. A computing device 140 in communication with the transceiver 120 can be used to initiate the transmission 201 of the FMCW signal 121. In step 202, a second signal 122 (reflected signal) from the subject 130 is received by the transceiver 120. In step 203, the transceiver transmits the second signal 122 data to the computing device 140. The transmission 203 can occur via a hard-wiring system or a wireless system such as WiFi, Bluetooth, and the like. In step 204, software 150 installed on the computing device 140 analyzes the second signal 122 data to determine types of physiological motion measured of the subject 130. In step 205, software 150 installed on the computing device processes the second signal 122 via an analog to digital converter (ADC). As will be understood by a person of ordinary skill in the art, the transmitted FMCW signal is modulated and the reflected signal must be demodulated. The reflected signal is demodulated using quadrature local oscillators to create I (“in-phase”) and Q (“quadrature”) data streams. In step 206, the software 150 performs a first set of filtering I and Q data to remove unwanted signal artifacts such as noise. In step 207, the software performs a frequency filter to a vital sign range. The vital sign range is a range of frequencies generated by a physiological motion of a subject such as respiration or heartbeat. In step 208, the software 150 takes a range of Fast Fourier Transforms (FFT). In step 209, the software 150 performs a target detection accuracy algorithm. In step 210, the software 150 fixes a Doppler offset. In step 211, the software 150 takes a velocity FFT. In step 212, the software 150 takes an inverse FFT. In step 213, the software 150 stores data to a file. Preferably, the software 150 stores between 10 to 15 seconds of data measurements in a file, although greater or fewer time intervals can be stored. In step 214, the software 150 performs smoothing filters to clean up the second signal, resulting in a motion signal corresponding to the physiological motion of the subject. In step 215, the software 150 calculates a rate of the physiological motion of the subject based on the motion signal. In step 216, the software 150 stores data corresponding to the calculated rate of the physiological motion of the subject.

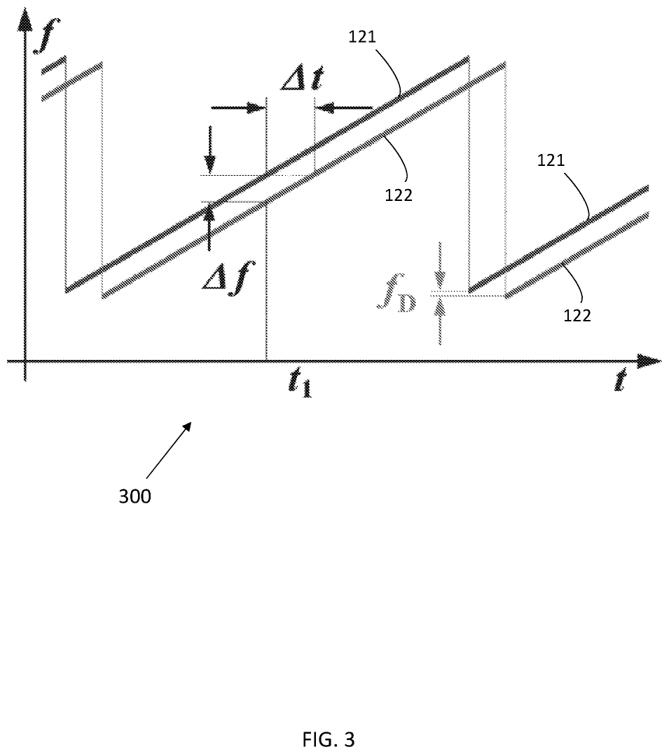

FIG. 3 illustrates a graph 300 depicting changes in frequency over time. The aforementioned FMCW radar systems and methods illustrated in flow diagrams 10 and 20 are a different approach for detecting objects. In contrast to pulse radar, the present invention has a permanent EM wave that is continuously transmitted. The frequency of the EM wave changes as a function of time. As shown, the transmitted signal has a time delay so that the reflected (also delayed) and the instantaneous transmit signal show slightly different instantaneous frequency values, as the transmit frequency changes to a different value. Changes in frequency over time is depicted as a sawtooth modulation in FIG. 3. The graph 300 is shown as a non-limiting example while other graphs can also be implemented to depict different frequency-time functions.

If using a 24 GHz FMCW radar transceiver, the RF signal bandwidth is 250 MHz and frequency deviation is allowed. Simple processing enables the transceiver and the subject to be as close as two or three meters apart (minimal distance). In order to have the transceiver closer to the subject, complex and fast digital signal processing (DSP) is required. At this lower distance to the subject, only one object can be detected. However, the present invention FMCW radar has a large zone of unambiguity, since the sawtooth repetition time can be selected as high as required.

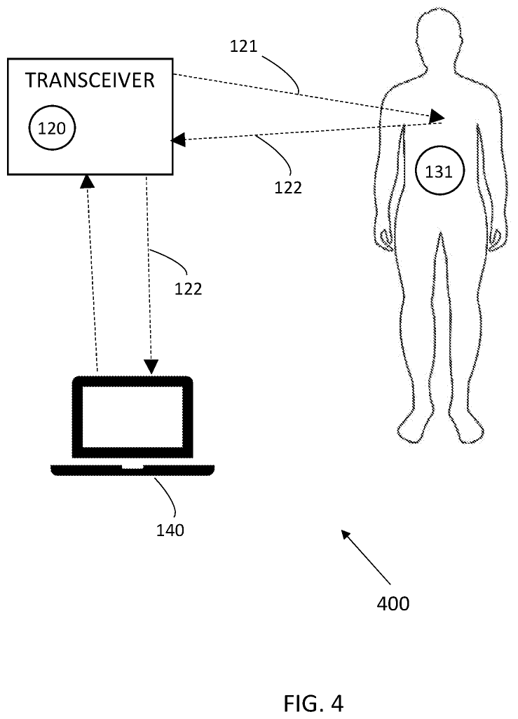

FIG. 4 illustrates a diagram 40 of a system and method for measuring physiological motion of a human subject 131. A computing device 140 in communication with a transceiver 120 can be used to initiate the transmission 101 of the FMCW signal 121. The transceiver 120 transmits the FMCW signal 121 toward the human subject 131. After reaching the human subject 131, a second signal 122 (reflected signal) from the subject 131 is received by the transceiver 120. The transceiver 120 transmits the second signal 122 data to the computing device 140. The transmission of the second signal 122 to the computing device 140 can occur via a hard-wiring system or a wireless system such as WiFi, Bluetooth, and the like.

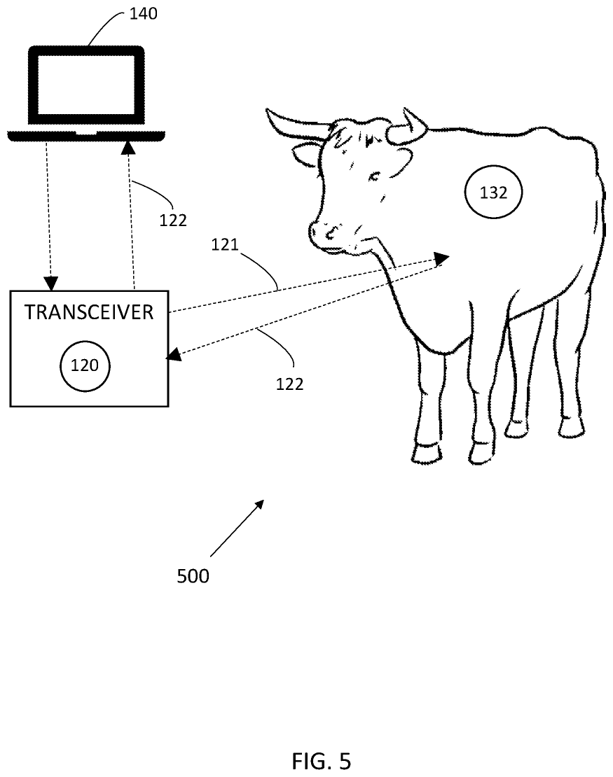

FIG. 5 illustrates a diagram 50 of a system and method for measuring physiological motion of an animal 132, namely cattle. The use of the term “animal” refers to a “non-human” animal in this disclosure. Having cattle as a subject is just one example of an animal whose physiological motion can be measured by the present invention. A computing device 140 in communication with a transceiver 120 can be used to initiate the transmission 101 of the FMCW signal 121. The transceiver 120 transmits the FMCW signal 121 toward the animal subject 132. After reaching the animal subject 132, a second signal 122 (reflected signal) from the subject 132 is received by the transceiver 120. The transceiver 120 transmits the second signal 122 to the computing device 140. The transmission of the second signal 122 to the computing device 140 can occur via a hard-wiring system or a wireless system such as WiFi, Bluetooth, and the like.

1. A method for detecting and measuring a physiological motion of a subject without contact with the subject, the method comprising:
a. generating a first signal, using a transceiver, wherein the first signal comprises a frequency modulated continuous wave (FMCW) signal;
b. transmitting the first signal towards the subject using the transceiver;
c. receiving a second signal from the subject using the transceiver, wherein the second signal comprises a reflection of the first signal;
d. transmitting the second signal to a computing device;
e. analyzing the second signal, using software installed on the computing device;
f. isolating, using the software, a motion signal corresponding the physiological motion of the subject;
g. calculating, using the software, a rate of the physiological motion of the subject based on the motion signal; and
h. storing data corresponding to the calculated rate of the physiological motion of the subject.

2. The method of claim 1, wherein the subject is a human.

3. The method of claim 1, wherein the subject is an animal.

4. The method of claim 1, wherein the second signal is wirelessly transmitted to the computing device.

5. The method of claim 4 wherein the second signal is wirelessly transmitted to the computing device via Wi-Fi.

6. The method of claim 4, wherein the second signal is wirelessly transmitted to the computing device via Bluetooth.

7. The method of claim 4, wherein the second signal transmitted to the computing device a hard-wiring system.

8. The method of claim 1, wherein the physiological motion of the subject comprises a heartbeat.

9. The method of claim 1, wherein the physiological motion of the subject comprises respiration.

10. A system for detecting and measuring motion of a subject without contact with the subject, the system comprising:
a. a transceiver;
b. wherein the transceiver is configured to generate a first signal, wherein the first signal comprises a frequency modulated continuous wave (FMCW) signal;
c. wherein the transceiver is further configured to transmit the first signal towards the subject;
d. wherein the transceiver is further configured to receive a second signal from the subject, wherein the second signal comprises a reflection of the first signal;
e. wherein the transceiver is further configured to transmit the second signal to a computing device;
f. software installed on the computing device;
g. wherein the software is configured to analyze the second signal;
h. wherein the software is further configured to isolate a motion signal corresponding to a physiological motion of the subject;
i. wherein the software is further configured to calculate a rate of the physiological motion of the subject based on the motion signal; and
j. wherein the software is further configured to store data corresponding to the calculated rate of the physiological motion of the subject.

11. A method for detecting and measuring a physiological motion of a subject without contact with the subject, the method comprising:
a. generating a first signal, using a transceiver, wherein the first signal comprises a frequency modulated continuous wave (FMCW) signal;
b. transmitting the first signal towards the subject using the transceiver;
c. receiving a second signal from the subject using the transceiver, wherein the second signal comprises a reflection of the first signal;
d. transmitting the second signal to a computing device;
e. analyzing the second signal, using software installed on the computing device;
f. processing signal via an analog to digital converter (ADC) data using the software;
g. performing, using the software, a first set of filtering I and Q data;
h. performing, using the software, a frequency filter to a vital sign range;
i. taking, using the software, a range of fast Fourier Transforms (FFT);
j. performing, using the software, a target detection accuracy algorithm;
k. fixing, using the software, a Doppler offset;
l. taking, using the software, a velocity FFT;
m. taking, using the software, an inverse FFT;
n. storing, using the software, data to a file;
o. performing, using the software, smoothing filters to clean up the second signal and obtain a motion signal corresponding to the physiological motion of the subject;
p. calculating, using the software, a rate of the physiological motion of the subject based on the motion signal; and
q. storing, using the software, data corresponding to the calculated rate of the physiological motion of the subject.

12. The method of claim 11, wherein the subject is a human.

13. The method of claim 11, wherein the subject is an animal.

14. The method of claim 11, wherein the second signal is wirelessly transmitted to the computing device.

15. The method of claim 14 wherein the second signal is wirelessly transmitted to the computing device via Wi-Fi.

16. The method of claim 14 wherein the second signal is wirelessly transmitted to the computing device via Bluetooth.

17. The method of claim 14 wherein the second signal transmitted to the computing device via a hard-wired system.

18. The method of claim 11, wherein the physiological motion of the subject comprises a heartbeat.

19. The method of claim 11, wherein the physiological motion of the subject comprises respiration.
